SEQUENCE LISTING (8 sequences)

SEQ ID NO: 1 - protein, 12 aa, Artificial
  GE11
YHWYGYTPQNVI

SEQ ID NO: 2 - protein, 13 aa, Artificial
  GE11C
YHWYGYTPQNVIC

SEQ ID NO: 3 - RNA, 19 nt, Artificial
  siRNA sense strand
  misc_feature (1)..(6): 2'-O-methyl group on each nucleotide
  misc_feature (7)..(7): 2'-fluoro group
  misc_feature (8)..(8): 2'-O-methyl group
  misc_feature (9)..(11): 2'-fluoro group on each nucleotide
  misc_feature (12)..(19): 2'-O-methyl group on each nucleotide
gucucuuggauauucucga

SEQ ID NO: 4 - RNA, 21 nt, Artificial
  siRNA antisense strand
  misc_feature (1)..(1): 2'-O-methyl group
  misc_feature (2)..(2): 2'-fluoro group
  misc_feature (3)..(5): 2'-O-methyl group on each nucleotide
  misc_feature (6)..(6): 2'-fluoro group
  misc_feature (7)..(13): 2'-O-methyl group on each nucleotide
  misc_feature (14)..(14): 2'-fluoro group
  misc_feature (15)..(15): 2'-O-methyl group
  misc_feature (16)..(16): 2'-fluoro group
ucgagaauauccaagagacag

SEQ ID NO: 5 - RNA, 19 nt, Artificial
  siRNA sense strand
  misc_feature (1)..(6): 2'-O-methyl group on each nucleotide
  misc_feature (7)..(7): 2'-fluoro group
  misc_feature (8)..(8): 2'-O-methyl group
  misc_feature (9)..(11): 2'-fluoro group on each nucleotide
  misc_feature (12)..(19): 2'-O-methyl group on each nucleotide
cagcuaauucagaaucauu

SEQ ID NO: 6 - RNA, 21 nt, Artificial
  siRNA antisense strand
  misc_feature (1)..(1): 2'-O-methyl group
  misc_feature (2)..(2): 2'-fluoro group
  misc_feature (3)..(5): 2'-O-methyl group on each nucleotide
  misc_feature (6)..(6): 2'-fluoro group
  misc_feature (7)..(13): 2'-O-methyl group on each nucleotide
  misc_feature (14)..(14): 2'-fluoro group
  misc_feature (15)..(15): 2'-O-methyl group
  misc_feature (16)..(16): 2'-fluoro group
  misc_feature (17)..(21): 2'-O-methyl group on each nucleotide
aaugauucugaauuagcugua

SEQ ID NO: 7 - RNA, 21 nt, Artificial
  siRNA sense strand
  misc_feature (1)..(1): 2'-fluoro group
  misc_feature (2)..(2): 2'-O-methyl group
  misc_feature (3)..(3): 2'-fluoro group
  misc_feature (4)..(4): 2'-O-methyl group
  misc_feature (5)..(5): 2'-fluoro group
  misc_feature (6)..(6): 2'-O-methyl group
  misc_feature (7)..(7): 2'-fluoro group
  misc_feature (8)..(8): 2'-O-methyl group
  misc_feature (9)..(11): 2'-fluoro group on each nucleotide
  misc_feature (12)..(12): 2'-O-methyl group
  misc_feature (13)..(13): 2'-fluoro group
  misc_feature (14)..(14): 2'-O-methyl group
  misc_feature (15)..(15): 2'-fluoro group
  misc_feature (16)..(16): 2'-O-methyl group
  misc_feature (17)..(17): 2'-fluoro group
  misc_feature (18)..(18): 2'-O-methyl group
  misc_feature (19)..(19): 2'-fluoro group
  misc_feature (20)..(20): 2'-O-methyl group
  misc_feature (21)..(21): 2'-fluoro group
guaguuggagcuggugacgua

SEQ ID NO: 8 - RNA, 23 nt, Artificial
  siRNA antisense strand
  misc_feature (1)..(1): 2'-O-methyl group
  misc_feature (2)..(2): 2'-fluoro group
  misc_feature (3)..(3): 2'-O-methyl group
  misc_feature (4)..(4): 2'-fluoro group
  misc_feature (5)..(5): 2'-O-methyl group
  misc_feature (6)..(6): 2'-fluoro group
  misc_feature (7)..(7): 2'-O-methyl group
  misc_feature (8)..(8): 2'-fluoro group
  misc_feature (9)..(9): 2'-O-methyl group
  misc_feature (10)..(10): 2'-fluoro group
  misc_feature (11)..(13): 2'-O-methyl group on each nucleotide
  misc_feature (14)..(14): 2'-fluoro group
  misc_feature (15)..(15): 2'-O-methyl group
  misc_feature (16)..(16): 2'-fluoro group
  misc_feature (17)..(17): 2'-O-methyl group
  misc_feature (18)..(18): 2'-fluoro group
  misc_feature (19)..(19): 2'-O-methyl group
  misc_feature (20)..(20): 2'-fluoro group
  misc_feature (21)..(23): 2'-O-methyl group on each nucleotide
uacgucaccagcuccaacuacca

(19) United States
(12) Patent Application Publication
(10) Pub. No.: US 2023/0285579 A1
(43) Pub. Date: Sep. 14, 2023

(54) NUCLEIC ACID LIGAND CONJUGATES AND USE THEREOF FOR DELIVERY TO CELLS

(71) Applicant: The University of North Carolina at Chapel Hill, Chapel Hill, NC (US)

(72) Inventors: Chad Pecot, Pittsboro, NC (US); Salma H. Azam, Cary, NC (US); Albert Bowers, Chapel Hill, NC (US); Matthew Cyril Fleming, Canboro, NC (US)

(21) Appl. No.: 17/997,398

(22) PCT Filed: May 12, 2021
(86) PCT No.: PCT/US2021/031903

§ 371 Date: Oct. 28, 2022

Related U.S. Application Data
(60) Provisional application No. 63/024,142, filed on May 13, 2020.

(51) Int. Cl. A61K 47/64; A61K 47/54; A61P 35/00; C12N 15/113

(52) U.S. Cl. CPC A61K 47/64; A61K 47/549; A61P 35/00; C12N 15/1135; C12N 2310/14; C12N 2310/3513

(57) ABSTRACT

The present invention relates to conjugated products comprising a ligand linked to a nucleic acid. The invention further relates to methods for delivering a nucleic acid to a cell and treating a disease using the conjugated products. The invention further relates to methods of increasing uptake of a nucleic acid by a cell comprising conjugating the nucleic acid to a ligand to form the conjugated product of the invention.

### STATEMENT OF PRIORITY

This application claims the benefit, under 35 U.S.C. § 119(e), of U.S. Provisional Application No. 63/024,142, filed on May 13, 2020, the entire contents of which are incorporated by reference herein.

### FIELD OF THE INVENTION

The present invention relates to conjugated products comprising a ligand linked to a nucleic acid. The invention further relates to methods for delivering a nucleic acid to a cell and treating a disease using the conjugated products. The invention further relates to methods of increasing uptake of a nucleic acid by a cell comprising conjugating the nucleic acid to a ligand to form the conjugated product of the invention.

### BACKGROUND OF THE INVENTION

Difficulties with blocking many important cancer targets using conventional approaches have prompted the inventors and others to consider using RNA interference (RNAi) as a therapeutic approach (Pecot et al., Nat. Rev. Cancer 11:59 (2011)). The 1998 discovery of RNAi (Fire et al., Nature 391:806 (1998)), which led to the 2006 Nobel Prize, revealed that double-stranded RNAs (dsRNAs) can bind an mRNA sequence within the cytoplasm and subsequently cause the mRNA to be either degraded or translationally repressed by the RNA-induced silencing complex (RISC) (Pecot et al., Nat. Rev. Cancer 11:59 (2011)). Given the ability of RNAi to potently silence any RNA sequence, the use of “therapeutic RNAi” to target “undruggable” targets is highly desirable. The inventors have previously demonstrated the proof-of-concept that RNAi can be used to target mutant KRAS (Pecot et al., Mol. Cancer Ther. 13:2876 (2014)).

Although the use of RNAi has tremendous potential, many practical challenges stand in the way of successful and efficient delivery of oligonucleotides for cancer therapies (Pecot et al., Nat. Rev. Cancer 11:59 (2011)). Obstacles include intravascular degradation by serum exo- and endo-nucleases, rapid oligonucleotide clearance, the need for endosomal escape, and avoidance of immune stimulation. Over the past several years, the use of targetable, biocompatible ligands (e.g., GalNAC to target the asialoglycoprotein receptor (ASGPR)) and that of state-of-the-art chemically modified siRNAs (siRs) have shown some success in circumventing many of these barriers (Foster et al., Mol. Ther. 26:708 (2018); Nair et al., J. Am. Chem. Soc. 136:16958 (2014)). Given the challenges described, many have turned to use of nanoparticle carriers to delivery oligonucleotides to tumors, however thus far that has not led to clinical success and there are no FDA-approved therapies for nanoparticle-mediated oligonucleotide delivery in cancer.

Despite advances made in the art, there remains a need in the art for improved systems enabling delivery of nucleic acids to cells, e.g., cancer cells. Accordingly, the present invention overcomes the deficiencies in the art by providing compositions and methods using targeting ligands to deliver a nucleic acid to a cell.

### SUMMARY OF THE INVENTION

Owing to the remarkable success of GalNAC-conjugated chemically optimized siRs such as FDA-approved givosiran (Sardh et al., N. Engl. J. Med. 380:549 (2019)), which targets ASGPR in the liver, the inventors chose to evaluate whether a ligand-conjugated approach could target cancer and obviate the need for a nano-carrier. It was found that the epidermal growth factor receptor (EGFR) is highly expressed in most epithelial cancers using the cancer cell line encyclopedia (CCLE) dataset. The GE11 ligand was chosen, which is a 12-amino acid peptide that was discovered to bind EGFR but does not induce mitogenic signaling (Li et al., FASEB J. 19:1978 (2005)). A highly scalable, facile GE11 synthesis and click chemistry approach was developed to conjugate GE11 to siRNAs using a biocompatible polyethylene glycol (PEG) linker. In two cell lines from different carcinoma origins (LU65-lung cancer, HCT116-colon cancer), both of which are EGFR-expressing, compared with unconjugated siRs, GE11-Cy5-siRNAs were rapidly and abundantly internalized into the cells. Substantial RNAi activity was also observed when using GE11-KRAS targeting siRNAs without using transfection reagents. The degree of uptake of the siRNAs into both cell lines, which reached approximately >150-250 fold increase in just 24 hours, is remarkable and far beyond what would have been expected considering the levels (~8-fold increase) seen when using GalNAC-siRNAs designed to target hepatocytes (Nair et al., J. Am. Chem. Soc. 136:16958 (2014)). Given the degree of oligonucleotide uptake into cancer cells, ligand-conjugated nucleic acid delivery has real potential for using nucleic acids as a therapeutic in cancer.

Thus, one aspect of the invention relates to a conjugated product comprising:
a) a polypeptide comprising an epidermal growth factor receptor (EGFR) targeting moiety;
b) a linker; and
c) a nucleic acid.

Another aspect of the invention relates to a composition, e.g., a pharmaceutical composition, comprising the conjugated product of the invention.

A further aspect of the invention relates to a method of delivering a nucleic acid into a cell, the method comprising contacting the cell with an effective amount of the conjugated product or composition of the invention.

An additional aspect of the invention relates to a method of treating a disease in a subject in need thereof, the method comprising administering to the subject a therapeutically effective amount of the conjugated product or pharmaceutical composition of the invention, thereby treating the disease.

Another aspect of the invention relates to a method of increasing uptake of a nucleic acid by a cell, the method comprising conjugating the nucleic acid through a linker to a polypeptide comprising an EGFR targeting moiety to form a conjugated product, wherein the cell expresses EGFR and wherein the uptake of the nucleic acid by the cell is increased relative to a nucleic acid that has not been conjugated to a polypeptide comprising an EGFR targeting moiety.

These and other aspects of the invention are set forth in more detail in the description of the invention below.

### BRIEF DESCRIPTION OF THE DRAWINGS

FIG. 1 shows many carcinomas express high levels of the EGFR.

FIG. 2 shows LU65 lung cancer cell lines have high EGFR expression confirmed by FACS.

FIG. 3 shows HCT116 colon cancer cell lines have high EGFR expression confirmed by FACS.

FIG. 4 shows the synthesis scheme for the conjugated product.

FIG. 5 shows LC/MS confirmation of conjugation products for GE11-PEG-KRAS Seq3 siRNA.

FIG. 6 shows LC/MS confirmation of conjugation products for GE11-PEG-KRASSeq3 siRNA.

FIG. 7 shows GE11-conjugated Cy5-labeled siRNAs show a dramatic time-dependent free uptake (no transfection reagent) into two different EGFR-expressing cancer cells (colon and lung cancer).

FIG. 8 shows GE11-conjugated KRAS silencing siRNAs (Seq2 and Seq3) provide significant KRAS silencing at 48 hours for both siRNAs in HCT116 (KRAS G13D mutant) colon cancer cells. No transfection reagent was used. This demonstrates GE11-conjugated siRNAs can be taken up into cells and effectively silence an mRNA.

FIG. 9 shows GE11-conjugated Cy5-labeled siRNAs enter cells through a receptor-mediated endocytosis mechanism in EGFR-expressing cancer cells (HCT116 colon cancer). Cancer cells were first transfected with GFP-labeled reporter plasmids that localize to either early (green, Rab5a) and late (green, Rab7a) endosomes or lysosome (green, Lamp1) subcellular structures. The cells were then treated without a transfection reagent with GE11-siRNAs in the culture media for 4 hours. The cells were then washed three times with PBS and were then imaged. The Cy5 signal (cyan) co-localizes with early and late endosomes as well as lysosomes, indicating they enter cells through a receptor-mediated endocytosis mechanism.

FIG. 10 shows GE11-conjugated Cy5-labeled siRNAs enter cells through a receptor-mediated endocytosis mechanism in EGFR-expressing cancer cells (HCT116 colon cancer). Cancer cells were first transfected with GFP-labeled reporter plasmids that localize to either early (green, Rab5a) and late (green, Rab7a) endosomes or lysosome (green, Lamp1) subcellular structures. The cells were then treated without a transfection reagent with GE11-siRNAs in the culture media for 24 hours. The cells were then washed three times with PBS and were then imaged. The Cy5 signal (cyan) co-localizes with early and late endosomes as well as lysosomes, indicating they enter cells through a receptor-mediated endocytosis mechanism.

FIGS. 11A-11B show in vivo evidence for KRAS silencing in HCT116 tumors (KRAS G13D). HCT116 (KRAS G13D) tumors were established at ~125 mm³ in size, and then treated with either PBS or an EGFR-targeting ligand (GE11) conjugated to a KRAS siRNA sequence with the shown linkers. The linkers evaluated utilized a hexylamino linker conjugated with a cleavable disulfide bond (SPDP) or non-cleavable (SMCC or TEG) handle. (SPDP: succinimidyl 3-(2-pyridyldithio)propionate; SMCC: Succinimidyl-trans-4-(N-maleimidylmethyl)cyclohexane-1-carboxylate; TEG: triethylene glycol). The mice were treated with 5 mg/kg (mpk) subcutaneously with the GE11-siRNAs suspended in sterile PBS. Tumors were extracted at 3 (D3) and 7 (D7) days after a single subcutaneous injection of the GE11-siRNAs injected in 200 µL/mouse of PBS. Tumor RNA was isolated and real-time qPCR was run for KRAS and 18S housekeeping gene. Up to 50-70% silencing of KRAS in the tumors was found at the indicated time-points. Each group and timepoint indicated represents 5 independent tumors. *p<0.05, ***p<0.01, ****p<0.001. Mice were also given one-time doses of 5 mpk or 10 mpk or 5 daily doses of 10 mpk (cumulative of 50 mpk in 5 days) and no observable toxicity or weight loss was found.

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage. See, e.g., PatentIn User Manual, 99-102 (November 1990) (U.S. Pat. and Trademark Office).

Except as otherwise indicated, standard methods known to those skilled in the art may be used for the construction of recombinant parvovirus and AAV (rAAV) constructs, packaging vectors expressing the parvovirus Rep and/or Cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, NY, 2012); AUSUBEL et al., CURRENT PROTOCOLS IN MOLECULAR BIOLOGY (Green Publishing Associates, Inc. and John Wiley & Sons, Inc., New York).

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps “and those that do not materially affect the basic and novel characteristic(s)” of the claimed invention (e.g., nucleic acid delivery). Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to modify the expression of a target nucleic acid of at least about 50% or more as compared to the expression level of a polynucleotide consisting of the recited sequence. The term “materially altered,” as applied to polypeptides of the invention, refers to an increase or decrease in enzymatic activity of at least about 50% or more as compared to the activity of a polypeptide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, the term “polypeptide” encompasses both peptides and proteins, unless indicated otherwise.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

As used herein, an “isolated” polynucleotide (e.g., an “isolated DNA” or an “isolated RNA”) means a polynucleotide separated or substantially free from at least some of the other components of the naturally occurring organism or virus, for example, the cell or viral structural components or other polypeptides or nucleic acids commonly found associated with the polynucleotide.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

The term “fragment,” as applied to a polynucleotide, will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell’s descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

As used herein, the terms “contacting,” “introducing” and “administering” are used interchangeably, and refer to a process by which the conjugated product of the present invention or a polynucleotide of this invention is delivered to a cell, in order to inhibit or alter or modify expression of a target gene or cellular process. The conjugated product may be administered in a number of ways, including, but not limited to extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and mitochondrial genome, and therefore includes integration of the nucleic acid into, for example, the mitochondrial genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism. Stable transformation of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

As used herein, a “transfection reagent” is any compound or molecule which is used to enhance delivery of a nucleic acid into a cell, either by contacting the cell before and/or simultaneously contacting the cell with the nucleic acid or contacting the nucleic acid before the nucleic acid contacts the cell. A transfection reagent is not covalently bound to the nucleic acid.

A “therapeutic polypeptide” or “therapeutic nucleic acid” is a polypeptide or nucleic acid that may alleviate or reduce symptoms that result from an absence, insufficient or excesses levels, or defect in a protein or nucleic acid in a cell or subject. Alternatively, a “therapeutic polypeptide” is one that otherwise confers a benefit to a subject, e.g., anti-cancer effects or improvement in transplant survivability.

As used herein, “RNAi” or “RNA interference” refers to the process of sequence-specific post-transcriptional gene silencing, mediated by double-stranded RNA (dsRNA). As used herein, “dsRNA” refers to RNA that is partially or completely double stranded. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism. After introduction into the organism, the target gene-specific dsRNA is processed into relatively small fragments (siRNAs) and can subsequently become distributed throughout the organism, leading to a loss-of-function mutation having a phenotype that, over the period of a generation, may come to closely resemble the phenotype arising from a complete or partial deletion of the target gene.

MicroRNAs (miRNAs) are non-protein coding RNAs, generally of between about 18 to about 25 nucleotides in length. These miRNAs direct cleavage in trans of target transcripts, negatively regulating the expression of genes involved in various regulation and development pathways (Bartel, Cell 116:281-297 (2004); Zhang et al., Dev. Biol. 289:3-16 (2006)). As such, miRNAs have been shown to be involved in different aspects of growth and development as well as in signal transduction and protein degradation. Since the first miRNAs were discovered in plants (Reinhart et al., Genes Dev. 16:1616-1626 (2002), Park et al.. Curr. Biol. 12:1484-1495 (2002)) many hundreds have been identified. Many microRNA genes (MIR genes) have been identified and made publicly available in a database (miRBase; microma.sanger.ac.uk/sequences). miRNAs are also described in U.S. Pat. Publications 2005/0120415 and 2005/144669A1, the entire contents of which are incorporated by reference herein.

Genes encoding miRNAs yield primary miRNAs (termed a “pri-miRNA”) of 70 to 300 bp in length that can form imperfect stem loop structures. A single pri-miRNA may contain from one to several miRNA precursors. In animals, pri-miRNAs are processed in the nucleus into shorter hairpin RNAs of about 65 nt (pre-miRNAs) by the RNaselll enzyme Drosha and its cofactor DGCR8/Pasha. The pre-miRNA is then exported to the cytoplasm, where it is further processed by another RNaseIII enzyme, Dicer, releasing a miRNA/miRNA* duplex of about 22 nt in size. Many reviews on microRNA biogenesis and function are available, for example, see, Bartel, Cell 116:281-297 (2004), Murchison et al., Curr. Opin. Cell Biol. 16:223-229 (2004), Dugas et al., Curr. Opin. Plant Biol. 7:512-520 (2004) and Kim, Nature Rev. Mol. Cell Biol. 6:376-385 (2005).

As used herein, the term “modified,” as applied to a polynucleotide or polypeptide sequence, refers to a sequence that differs from a wild-type sequence due to one or more deletions, additions, substitutions, chemical modifications, or any combination thereof.

As used herein, by “isolate” or “purify” (or grammatical equivalents) a virus vector, it is meant that the virus vector is at least partially separated from at least some of the other components in the starting material.

By the terms “treat,” “treating,” or “treatment of” (and grammatical variations thereof) it is meant that the severity of the subject’s condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” or “therapeutically effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

### Conjugated Products

One aspect of the present invention relates to a conjugated product comprising:
a) a polypeptide comprising an epidermal growth factor receptor (EGFR) targeting moiety;
b) a linker; and
c) a nucleic acid.

The polypeptide comprising an EGFR targeting moiety may be any targeting moiety known in the art or later identified. In some embodiments, the polypeptide comprises, consists essentially of, or consist of the amino acid sequence of the dodecapeptide GE11 (YHWYGYTPQNVI (SEQ ID NO:1)) or a sequence at least 80% identical thereto, e.g., at least 80%, 85%, 90%, 91%, 92%, 93%, 94%, 95%, 96%, 97%, 98%, or 99% identical thereto. The GE11 sequence may be modified by any combination of additions, deletions and/or substitutions, and may include both naturally occurring and non-naturally occurring amino acids.

The modifications to the GE11 sequence or any other EGFR targeting moiety may be done to provide a reaction site for preparing the conjugated product. In one embodiment, the polypeptide is modified to comprise a cysteine residue at the C-terminus. For GE11, the additional cysteine residue forms the sequence YHWYGYTPQNVIC (SEQ ID NO:2)). In some embodiments, the polypeptide comprises, consists essentially of, or consist of the amino acid sequence of SEQ ID NO:2 or a sequence at least 80% identical thereto, e.g., at least 80%, 85%, 90%, 91%, 92%, 93%, 94%, 95%, 96%, 97%, 98%, or 99% identical thereto.

In some embodiments, EGFR targeting moiety may be a carborane-containing macrocyclic peptide such as CbaP5 and CbaP14 as described in Yin et al., J. Am. Chem. Soc. 141:19193 (2019), incorporated by reference herein in its entirety.

The linker may be any linker suitable for linking the polypeptide and the nucleic acid, either covalently or non-covalently. In some embodiments, the linker is a pharmaceutically acceptable linker, such as but not limited to a polyethylene glycol (PEG) linker, a reducible disulfide linker, an acid-labile oxime linker, a reactive-oxygen species (ROS)-sensitive boronate ester linker, a peptide linker, or a hydrazone linker. In some embodiments, the linker is a hexylamino linker conjugated with a cleavable disulfide bond (e.g., succinimidyl 3-(2-pyridyldithio)propionate (SPDP)) or non-cleavable (e.g., succinimidyl-trans-4-(N-maleimidylmethyl)cyclohexane-1-carboxylate (SMCC) or triethylene glycol (TEG)) handle.

In some embodiments, the linker may comprise polyethylene glycol (PEG). In some embodiments, the linker may comprise, consist essentially of, or consist of dibenzocyclooctyne-PEG4-maleimide.

In some embodiments, the polypeptide is covalently bound to the linker, e.g., covalently bound to the thiol group on the cysteine residue.

The nucleic acid may be any nucleic acid that is desired to be introduced into a cell, either in vitro or in vivo. The nucleic acid may be one that is useful for research or therapeutic purposes. The nucleic acid may be one that can be used to modify (increase or decrease) the level of a nucleic acid or protein in a cell.

In some embodiments, the nucleic acid is a DNA, a RNA, or a hybrid of DNA and RNA. In some embodiments, the nucleic acid is double stranded or single stranded. Where single-stranded, the nucleic acid can be a sense strand or an antisense strand.

The nucleic acid may be constructed using chemical synthesis and enzymatic ligation reactions by procedures known in the art. For example, a nucleic acid may be chemically synthesized using naturally occurring nucleotides or various modified nucleotides designed to increase the biological stability of the molecules or to increase the physical stability of the duplex formed between the nucleic acid and target nucleotide sequences, e.g., phosphorothioate derivatives and acridine substituted nucleotides can be used. Examples of modified nucleotides which can be used to generate the nucleic acid include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet- hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the nucleic acid can be produced using an expression vector into which a nucleic acid encoding the nucleic acid has been cloned.

The nucleic acid can further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every one or every other one of the internucleotide bridging phosphate residues can be modified as described. In another non-limiting example, the nucleic acid is a nucleotide sequence in which at least one, or all, of the nucleotides contain a 2′ lower alkyl moiety (e.g., C1-C4, linear or branched, saturated or unsaturated alkyl, such as methyl, ethyl, ethenyl, propyl, 1-propenyl, 2-propenyl, and isopropyl). In another example, one or more of the nucleotides may be a 2′-fluoro nucleotide, a 2—O-methyl nucleotide, or a locked nucleic acid nucleotide. For example, every one or every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sci. USA 85:5011 (1988); incorporated by reference herein in their entireties for their teaching of methods of making polynucleotide molecules, including those containing modified nucleotide bases).

In certain embodiments, the nucleic acid may be selected from the group consisting of siRNA, microRNA, shRNA, antisense nucleic acid, ribozyme, killer-tRNA, guide RNA, long non-coding RNA, anti-miRNA oligonucleotide, and plasmid DNA.

In one embodiment, the nucleic acid is a KRAS silencing siRNA or antisense oligonucleotide, such as is described in U.S. Pat. No. 10,619,159 or U.S. Publication No. 2020/0248185.

In certain embodiments, the linker is covalently bound to the nucleic acid. The nucleic acid may be modified to provide a binding site to the linker, e.g., to comprise an azide group as the binding site.

An additional aspect of the invention relates to a composition comprising the conjugated product of the invention and a carrier. In certain embodiments, the composition is a pharmaceutical composition comprising the conjugated product of the invention and a pharmaceutically acceptable carrier.

A further aspect of the invention relates to a method of increasing uptake of a nucleic acid by a cell, the method comprising conjugating the nucleic acid through a linker to a polypeptide comprising an EGFR targeting moiety to form a conjugated product, wherein the cell expresses EGFR and wherein the uptake of the nucleic acid by the cell is increased relative to a nucleic acid that has not been conjugated to a polypeptide comprising an EGFR targeting moiety.

The nucleic acid, linker, and EGFR targeting moiety may be any of those described above. The conjugate may be prepared by any method known in the art and as described above and in the examples.

### Methods of Use

Various methods are provided herein, employing the conjugates and/or compositions of this invention. Thus, one aspect of the invention relates to a method of delivering a nucleic acid into a cell, the method comprising contacting the cell with an effective amount of the conjugate or composition of the invention. The cell may be an in vitro, ex vivo, or in vivo cell. In some embodiments, the cell is a cancer cell. In some embodiments, the cancer cell is selected from the group consisting of non-small cell lung cancer cell, lung cancer cell, colon cancer cell, pancreas cancer cell, and blood cancer cell. In certain embodiments, the cell expresses EGFR, e.g., higher levels of EGFR relative to other cells. In one embodiment, the cell is a cancer cell that expresses a higher level of EGFR relative to non-cancerous cells from the same subject or relative to the average level of EGFR found in the general population.

In some embodiments, the method of increasing uptake of a nucleic acid by a cell does not comprise using a transfection reagent separate from the conjugate.

Another aspect of the invention relates to a method of treating a disease in a subject in need thereof, the method comprising administering to the subject a therapeutically effective amount of the conjugate or pharmaceutical composition of the invention, thereby treating the disease. The disease may be one in which the diseased cells express EGFR, e.g., higher levels of EGFR relative to other cells. In some embodiments, the disease is cancer, e.g., a cancer is selected from the group consisting of non-small cell lung cancer, lung cancer, colon cancer, pancreas cancer, and blood cancer. In one embodiment, the cancer comprises a mutant human KRAS gene comprising one or more of the missense mutations G12C, G12D, G12V, and G13D. A cancer comprising a mutant human KRAS gene comprising one or more of the missense mutations G12C, G12D, G12V, and G13D is a cancer, e.g., a tumor in which one or more cells express the mutant KRAS gene.

In some embodiments, the method of treating a disease does not comprise using a transfection reagent separate from the conjugate.

In one embodiment of each of these aspects, the subject may be one that has been diagnosed with the disease, e.g., cancer. In another embodiment, the subject may be one that is at risk of developing the disease, e.g., cancer (e.g., predisposed due to hereditary factors, smoking, viral infection, exposure to chemicals, etc.). In a further embodiment, the subject may be one that has been identified as carrying a mutant KRAS gene and has or has not been diagnosed with cancer.

The conjugate or composition of the invention can be delivered to a cell by contacting the cell using any method known in the art. In one embodiment, the conjugate or composition of the invention is administered directly to the subject. Generally, the conjugates of the invention will be suspended in a pharmaceutically-acceptable carrier (e.g., physiological saline) and administered orally, topically, or by intravenous infusion, or injected subcutaneously, intramuscularly, intracranially, intrathecally, intraperitoneally, intrarectally, intravaginally, intranasally, intragastrically, intratracheally, or intrapulmonarily. They are preferably delivered directly to the site of the disease or disorder, such as the lung, intestine, or pancreas. The conjugate or imposition can be delivered to a tumor by intratumoral injection or injection into a blood vessel feeding the tumor. The dosage required depends on the choice of the route of administration; the nature of the formulation; the nature of the patient’s illness; the subject’s size, weight, surface area, age, and sex; other drugs being administered; and the judgment of the attending physician. Suitable dosages are in the range of 0.01-100.0 µg/kg. Wide variations in the needed dosage are to be expected in view of the differing efficiencies of various routes of administration. For example, oral administration would be expected to require higher dosages than administration by i.v. injection (e.g., 2-, 3-, 4-, 6-, 8-, 10-; 20-, 50-, 100-, 150-, or more fold). Variations in these dosage levels can be adjusted using standard empirical routines for optimization as is well understood in the art. Administrations can be single or multiple. Encapsulation of the inhibitor in a suitable delivery vehicle (e.g., polymeric microparticles or implantable devices) may increase the efficiency of delivery, particularly for oral delivery.

The conjugate or composition of the present invention can optionally be delivered in conjunction with other therapeutic agents. The additional therapeutic agents can be delivered concurrently with the conjugate or composition of the invention. As used herein, the word “concurrently” means sufficiently close in time to produce a combined effect (that is, concurrently can be simultaneously, or it can be two or more events occurring within a short time period before or after each other). In one embodiment, the conjugate or composition of the invention are administered in conjunction with agents useful for treating cancer, such as: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxy progesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide). In another embodiment, the compounds of the invention are administered in conjunction with anti-angiogenesis agents, such as antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide TNF alpha conjugate, cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736, AMG-706, AZD2171, sorafenib tosylate, BMS-582664, CHIR-265, pazopanib, PI-88, vatalanib, everolimus, suramin, sunitinib malate, XL184, ZD6474, ATN-161, cilenigtide, and celecoxib, or any combination thereof.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms’ tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi’s sarcoma, polycythemia vera, essential thrombocytosis, Hodgkin’s disease, non-Hodgkin’s lymphoma, soft-tissue sarcoma, osteogenic sarcoma, primary macroglobulinemia, and retinoblastoma. In some embodiments, the cancer is selected from the group of tumor-forming cancers.

### Pharmaceutical Compositions

A further aspect of the invention relates to pharmaceutical formulations and methods of administering the same to achieve any of the therapeutic effects (e.g., treatment of cancer) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

[The conjugate or composition of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the conjugate (including the physiologically acceptable salts thereof) is typically admixed with, inter alia, an acceptable carrier. The carrier can be a solid or a liquid, or both, and is preferably formulated with the conjugate or composition as a unit-dose formulation, for example, a tablet, which can contain from 0.01 or 0.5% to 95% or 99% by weight of the conjugate or composition. One or more conjugate or composition can be incorporated in the formulations of the invention, which can be prepared by any of the well-known techniques of pharmacy.

A further aspect of the invention is a method of treating subjects in vivo, comprising administering to a subject a pharmaceutical composition comprising a conjugate or composition of the invention in a pharmaceutically acceptable carrier, wherein the pharmaceutical composition is administered in a therapeutically effective amount. Administration of the conjugate or composition of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular conjugate which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the conjugate can be administered in solid dosage forms, such as capsules, tablets, and powders, or in liquid dosage forms, such as elixirs, syrups, and suspensions. Conjugates can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric- coated for selective disintegration in the gastrointestinal tract. Liquid dosage forms for oral administration can contain coloring and flavoring to increase patient acceptance.

Formulations suitable for buccal (sub-lingual) administration include lozenges comprising the conjugate in a flavored base, usually sucrose and acacia or tragacanth; and pastilles comprising the conjugate in an inert base such as gelatin and glycerin or sucrose and acacia.

Formulations of the present invention suitable for parenteral administration comprise sterile aqueous and non-aqueous injection solutions of the conjugate, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a conjugate of the invention, in a unit dosage form in a sealed container. The conjugate or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the conjugate or salt. When the conjugate or salt is substantially water-insoluble, a sufficient amount of emulsifying agent which is pharmaceutically acceptable can be employed in sufficient quantity to emulsify the conjugate or salt in an aqueous carrier. One such useful emulsifying agent is phosphatidyl choline.

Formulations suitable for rectal administration are preferably presented as unit dose suppositories. These can be prepared by admixing the conjugate with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the conjugate. Suitable formulations comprise citrate or bis\tris buffer (pH 6) or ethanol/water and contain from 0.1 to 0.2 M of the conjugate.

The conjugate can alternatively be formulated for nasal administration or otherwise administered to the lungs of a subject by any suitable means, e.g., administered by an aerosol suspension of respirable particles comprising the conjugate, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the conjugate can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the conjugate can likewise be produced with any solid particulate medicament aerosol generator, by techniques known in the pharmaceutical art.

Alternatively, one can administer the conjugate in a local rather than systemic manner, for example, in a depot or sustained-release formulation.

Further, the present invention provides liposomal formulations of the conjugate disclosed herein and salts thereof. The technology for forming liposomal suspensions is well known in the art. When the conjugate or salt thereof is an aqueous-soluble salt, using conventional liposome technology, the same can be incorporated into lipid vesicles. In such an instance, due to the water solubility of the conjugate or salt, the conjugate or salt will be substantially entrained within the hydrophilic center or core of the liposomes. The lipid layer employed can be of any conventional composition and can either contain cholesterol or can be cholesterol-free. When the conjugate or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the conjugates disclosed herein or salts thereof, can be lyophilized to produce a lyophilizate which can be reconstituted with a pharmaceutically acceptable carrier, such as water, to regenerate a liposomal suspension.

In the case of water-insoluble conjugates, a pharmaceutical composition can be prepared containing the water-insoluble conjugate, such as for example, in an aqueous base emulsion. In such an instance, the composition will contain a sufficient amount of pharmaceutically acceptable emulsifying agent to emulsify the desired amount of the conjugate. Particularly useful emulsifying agents include phosphatidyl cholines and lecithin.

In particular embodiments, the conjugate is administered to the subject in a therapeutically effective amount, as that term is defined above. Dosages of pharmaceutically active compounds can be determined by methods known in the art, see, e.g., Remington’s Pharmaceutical Sciences (Maack Publishing Co., Easton, Pa). The therapeutically effective dosage of any specific compound will vary somewhat from compound to compound, and patient to patient, and will depend upon the condition of the patient and the route of delivery. As a general proposition, a dosage from about 0.001 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 µmol/kg to 50 µmol/kg, and more particularly to about 22 µmol/kg and to 33 µmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of a disease, e.g., cancer. In certain embodiments, the subject has or is at risk for a disease, e.g., cancer.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### Example 1

### Development of Nucleic Acid Conjugates

### Methods

Flow Cytometry: For EGFR expression profiling, detached LU65 or HCT116 cells were resuspended into a single cell solution in PBS. Fluorophore-conjugated EGFR or control IgG antibody was added to an aliquot of cell solution and incubated for 30 minutes on ice in the dark. Excess antibody was washed off the cells. Samples were then run on a flow cytometer to detect fluorescent signal and identify the proportion of cells that had been positively labeled by the antibody.

For assessment of free siRNA uptake, fluorescently-labeled siRNA (either unconjugated or conjugated to GE11) was incubated with LU65 or HCT116 cells for the indicated timepoints. Cells were then rinsed with PBS to wash away excess siRNA and then fixed in 2% paraformaldehyde. Samples were then run on a flow cytometer to detect fluorescent signal and identify the proportion of cells that had taken up fluorescent siRNA.

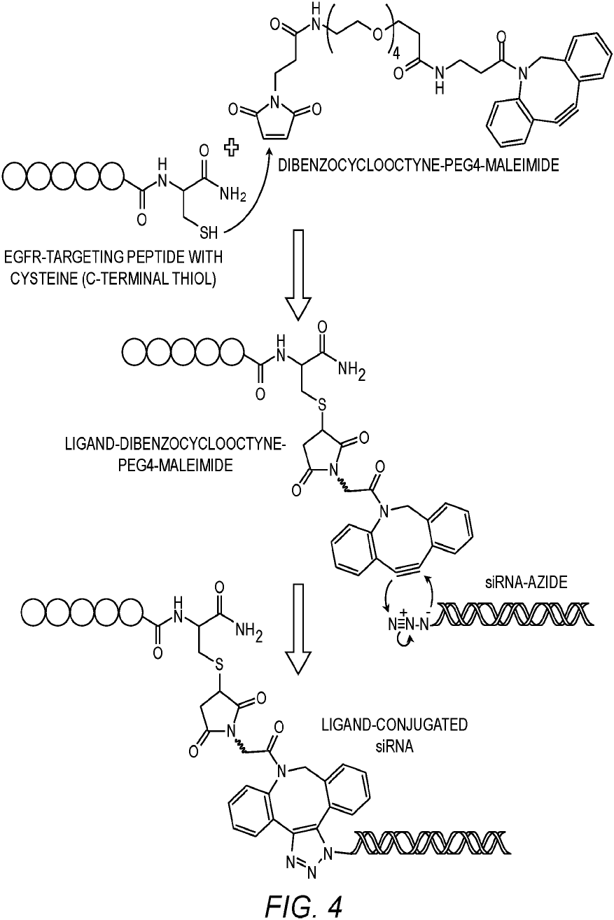

GE11 Synthesis and siRNA Conjugation: GE11 was synthesized using solid-phase peptide synthesis (FIG. 4). An extra cysteine amino acid was added to the end of the peptide chain (GE11C). The free thiol on the c-terminal cysteine was reacted with dibenzocyclooctyne (DBCO)-PEG4-maleimide reagent to generate a GE11C-DBCO-PEG4-maleimide conjugated product. A copper-free click reaction was performed to conjugate the DBCO-containing product with azide-conjugated siRNA to generate a final GE11C-siRNA conjugated product. The product was then run through an RNA Clean & Concentrator kit to remove excess unconjugated GE11 peptide.

Liquid Chromatography/Mass Spectrometry (LC/MS): GE11-conjugated siRNAs were analyzed by LC/MS to confirm successful conjugation of the peptide to the siRNA. LC/MS revealed the mass sizes of all species present in the submitted samples.

mRNA Expression Analysis: HCT116 or LU65 cells were incubated with control or GE11-conjugated anti KRAS siRNA for 48 hrs at the indicated doses. At 48 hours, media containing excess siRNA was removed and the cells were lysed in RNA lysis buffer. RNA was isolated from the lysate samples using an RNA isolation kit. RNA was then quantified using a Nanodrop spectrophotometer and equal amounts of RNA from each sample were loaded into a cDNA synthesis reaction. The resulting cDNA was analyzed by quantitative PCR using primers for KRAS and primers for a “housekeeping” gene which was used to normalize the results.

### Results

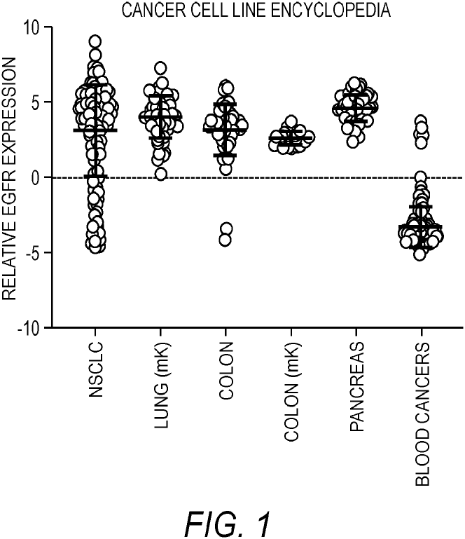

FIG. 1 demonstrates positive EGFR expression across hundreds of solid tumor-derived cancer cell lines, highlighting the potential for EGFR-mediated targeting of cancer.

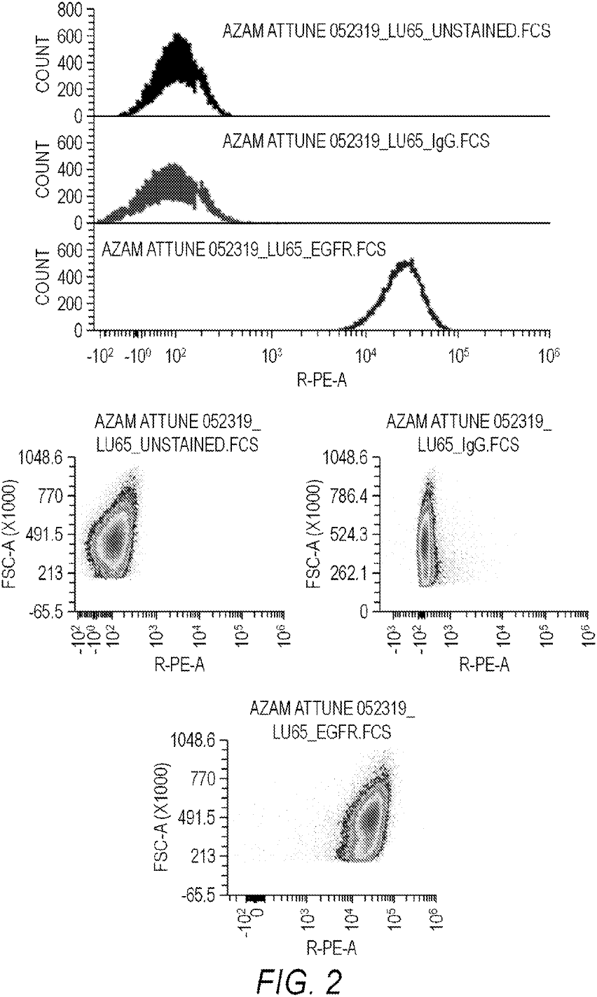

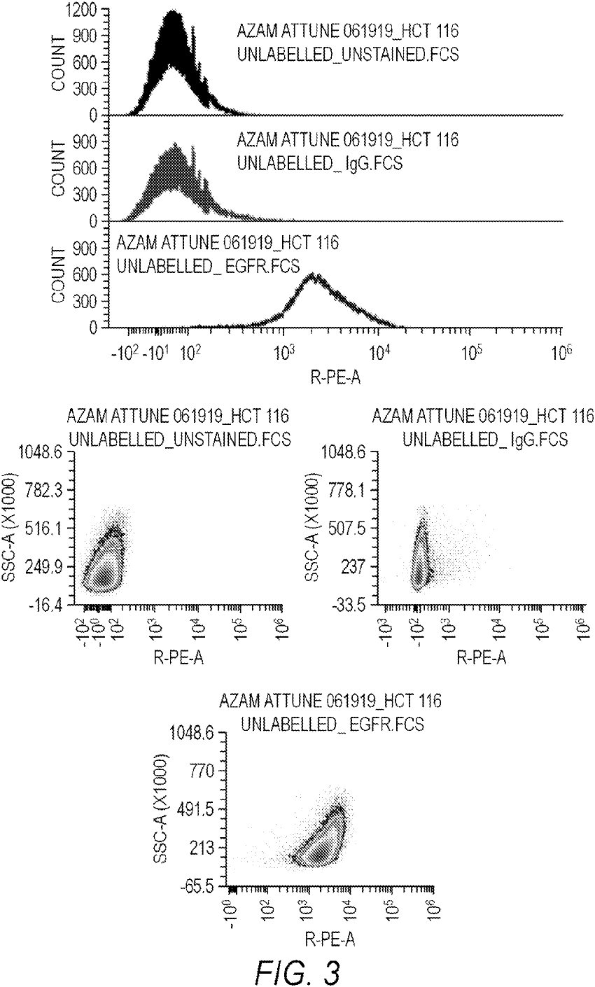

FIG. 2 and FIG. 3 show that staining either the lung cancer LU65 cell line or colon cancer HCT116 cell line (respectively) with an EGFR antibody results in a rightward shift (or increase) in fluorescent signal relative to cells stained with control IgG antibody. This indicates high EGFR positive expression in both cancer cell line models.

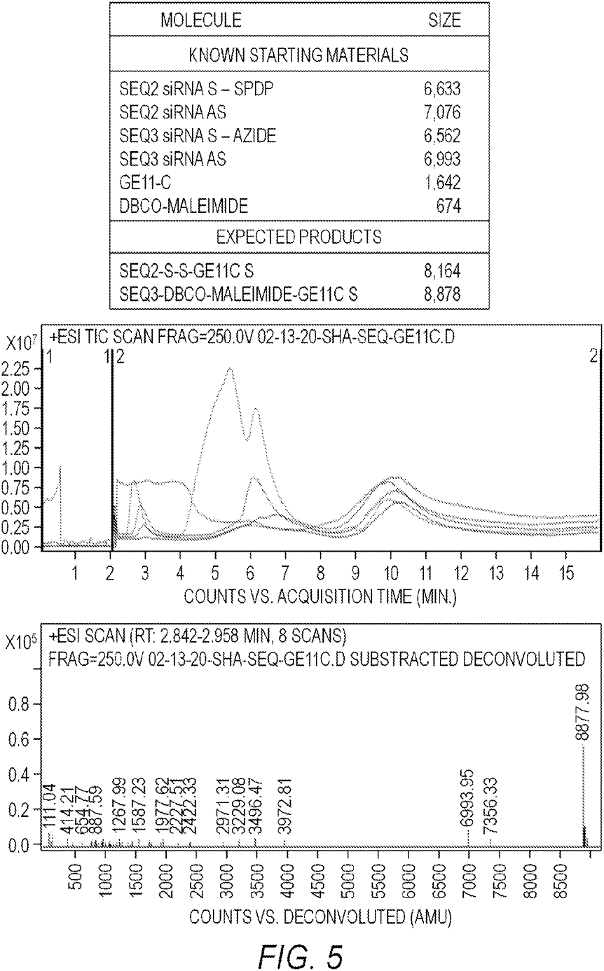

In FIG. 5, the presence of a mass product (8878) equal to the sum of the mass of GE11 (1642), the mass of the DBCO-PEG4-maleimide linker (674), and the mass of the antisense strand of the siRNA (6562) indicates the presence of successfully conjugated product.

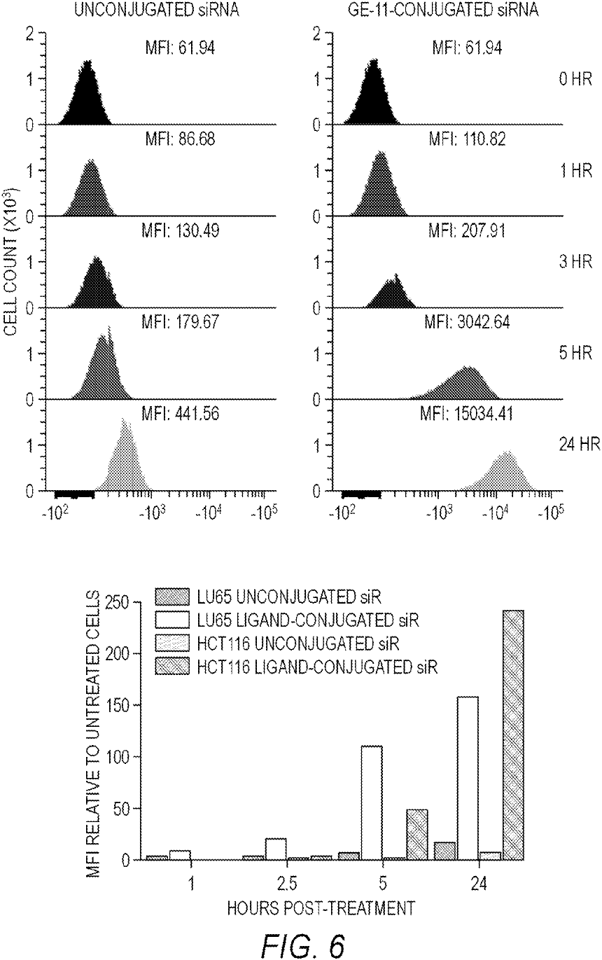

FIG. 6 demonstrates a dramatic increase over time of EGFR-expressing cancer cell uptake of fluorescently labeled siRNA when conjugated to GE11 relative to unconjugated fluorescently labeled siRNA. The siRNA used was Seq2-DV22 targeted to KRAS. These data suggest the presence of GE11 peptide significantly enhances siRNA delivery to cancer cells.

### Seq2-DV22

Sense strand (SEQ ID NO:3)

[mG]^{∗}[mU]^{∗}[mC][mU][mC][mU][2flU][mG][2flG][2flA][2
flU][mA][mU][mU][mC][mU][mC][mG][mA]

Antisense strand (SEQ ID NO:4)

[mU]^{∗}[2flC]^{∗}[mG][mA][mG][2flA][mA][mU][mA][mU][mC]
[mC][mA][2flA][mG][2flA][mG][mA][mC]^{∗}[mA]^{∗}[mG]

m - 2′-O-methyl on sugar moieties 2fl - 2′-fluoro on sugar moieties ^{∗}- phosphorothioate in between nucleotides

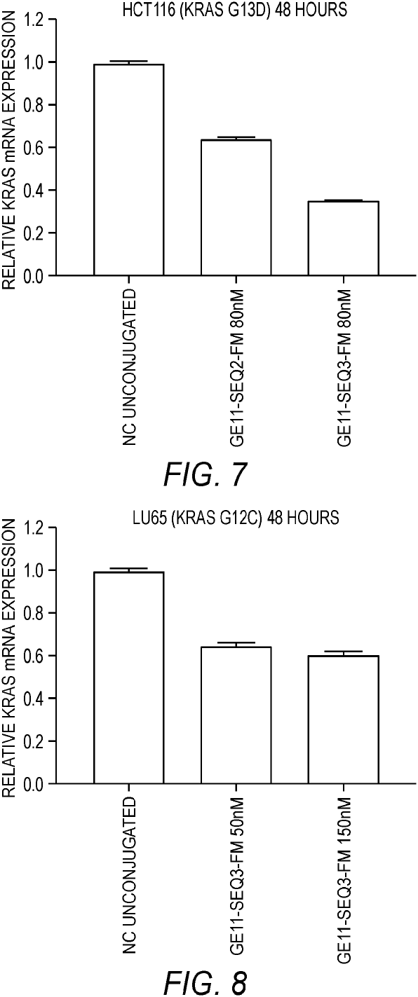

FIG. 7 and FIG. 8 demonstrate robust knockdown of target gene expression upon treatment with GE11-conjugated siRNA. The siRNAs used were Seq2—DV22 and Seq3—DV22 targeted to KRAS. Because no transfection reagent was used, siRNA entry into cells was completely dependent on receptor-mediated uptake. These data provide proof of principle that GE11-conjugation enables productive, efficacious uptake of siRNA which is functionally active upon cellular internalization.

### Seq3-DV22

Sense strand (SEQ ID NO:5)

[mC]^{∗}[mA]^{∗}[mG][mC][mU][mA][2flA][mU][2flU][2flC][2
flA][mG][mA][mA][mU][mC][mA][mU][mU]

Antisense strand (SEQ ID NO:6)

[mA]^{∗}[2flA]^{∗}[mU][mG][mA][2flU][mU][mC][mU][mG][mA]
[mA][mU][2flU][mA][2flG][mC][mU][mG]^{∗}[mU]^{∗}[mA]

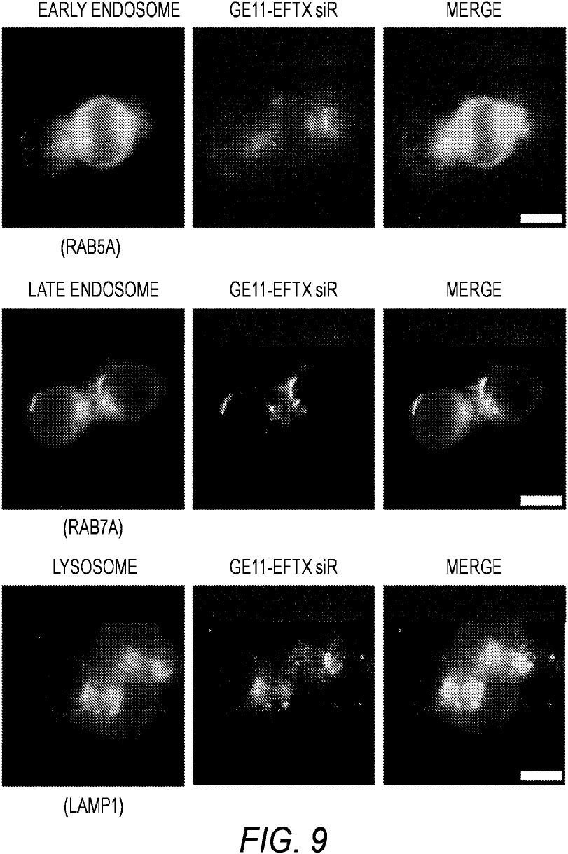

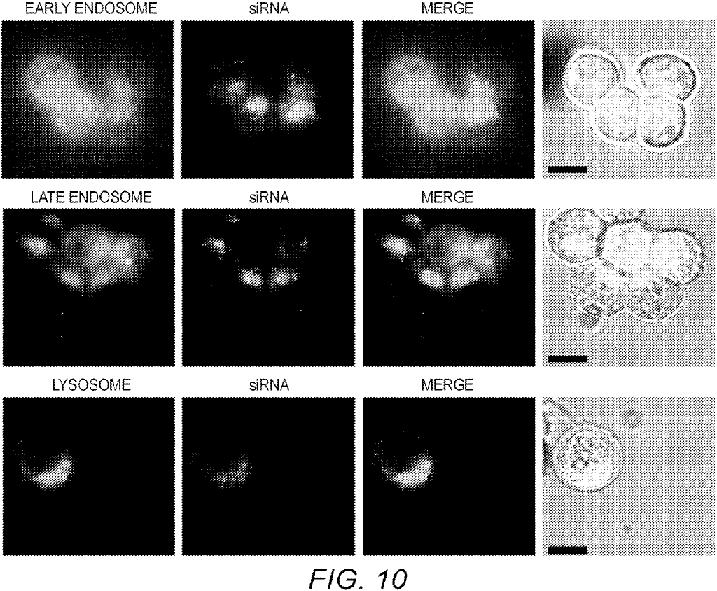

FIG. 9 and FIG. 10 demonstrate that GE11-conjugated Cy5-labeled siRNAs enter cells through a receptor-mediated endocytosis mechanism in EGFR-expressing cancer cells (HCT116 colon cancer). The siRNA used was Seq2-DV22 targeted to KRAS.

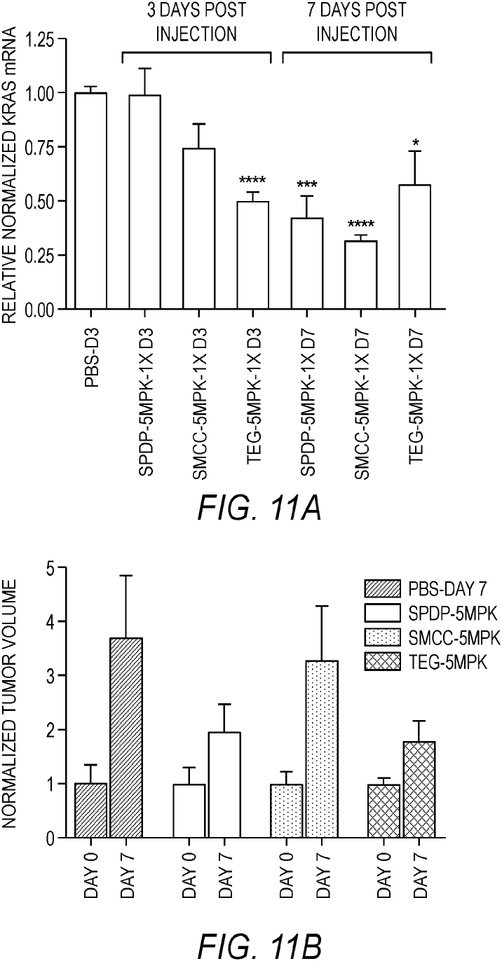

In vivo evidence for gene silencing using GE11-conjugated siRNAs is shown in FIGS. 11A-11B. HCT116 (KRAS G13D) tumors were established in mice, and then treated with either PBS or an EGFR-targeting ligand (GE11) conjugated to a KRAS siRNA sequence with the shown linkers (5 mg/kg). The siRNA used was D2-G13D-Hi2F targeted to KRAS. We found up to 50-70% silencing of KRAS in the tumors at the indicated time-points. Mice were also given one-time doses of 5 mg/kg or 10 mg/kg or 5 daily doses of 10 mg/kg (cumulative of 50 mg/kg in 5 days) and no observable toxicity or weight loss was found.

### D2-G13D-Hi2F

Sense strand (SEQ ID NO:7)

[2flG]^{∗}[mU]^{∗}[2flA][mG][2flU][mU][2flG][mG][2flA][2
flG][2flC][mU][2flG][mG][2flU][mG][2flA][mC][2flG]
[mU][2flA]

Antisense strand (SEQ ID NO:8)

[mU]^{∗}[2flA]^{∗}[mC][2flG][mU][2flC][mA][2flC][mC][2fl
A][mG][mC][mU][2flC][mC][2flA][mA][2flC][mU][2flA]
[mC]^{∗}[mC]^{∗}[mA]

The foregoing is illustrative of the present invention, and is not to be construed as limiting thereof. The invention is defined by the following claims, with equivalents of the claims to be included therein.

That which is claimed is:

1. A conjugated product comprising:
a) a polypeptide comprising an epidermal growth factor receptor (EGFR) targeting moiety;
b) a linker; and
c) a nucleic acid.

2. The conjugated product of claim 1, wherein the polypeptide comprises GE11.

3. The conjugated product of claim 1, wherein the polypeptide is modified to comprise a cysteine residue at the C-terminus.

4. The conjugated product of claim 1, wherein the polypeptide comprises the amino acid sequence of SEQ ID NO:2.

5. The conjugated product of claim 1, wherein the linker comprises polyethylene glycol (PEG).

6. The conjugated product of claim 1, wherein the linker comprises dibenzocyclooctyne-PEG4-maleimide.

7. The conjugated product of claim 1, wherein the linker is a hexylamino linker conjugated with a cleavable disulfide bond or non-cleavable handle.

8. The conjugated product of claim 1, wherein the linker is succinimidyl 3-(2-pyridyldithio)propionate, succinimidyl-trans-4-(N-maleimidylmethyl)cyclohexane-1-carboxylate, or triethylene glycol.

9. The conjugated product of claim 1, wherein the polypeptide is covalently bound to the linker.

10. The conjugated product of claim 9, wherein the polypeptide is covalently bound to the thiol group on the cysteine residue.

11. The conjugated product of claim 1, wherein the nucleic acid is a DNA.

12. The conjugated product of claim 1, wherein the nucleic acid is a RNA.

13. The conjugated product of claim 1, wherein the nucleic acid is single stranded.

14. The conjugated product of claim 1, wherein the nucleic acid is double stranded.

15. The conjugated product of claim 1, wherein the nucleic acid is selected from the group consisting of siRNA, microRNA, shRNA, antisense nucleic acid, ribozyme, killer-tRNA, guide RNA, long non-coding RNA, anti-miRNA oligonucleotide, and plasmid DNA.

16. (canceled)

17. The conjugated product of claim 1, wherein the linker is covalently bound to the nucleic acid.

18. The conjugated product of claim 17, wherein the nucleic acid is modified to provide a binding site to the linker.

19. The conjugated product of claim 18, wherein the nucleic acid is modified to comprise an azide group as the binding site.

20. (canceled)

21. A pharmaceutical composition comprising the conjugated product of claim 1 and a pharmaceutically acceptable carrier.

22. A method of delivering a nucleic acid into a cell, the method comprising contacting the cell with an effective amount of the composition of claim 21.

23-26. (canceled)

27. A method of treating a disease in a subject in need thereof, the method comprising administering to the subject a therapeutically effective amount of the pharmaceutical composition of claim 21, thereby treating the disease.

28-30. (canceled)

31. A method of increasing uptake of a nucleic acid by a cell, the method comprising conjugating the nucleic acid through a linker to a polypeptide comprising an EGFR targeting moiety to form a conjugated product, wherein the cell expresses EGFR and wherein the uptake of the nucleic acid by the cell is increased relative to a nucleic acid that has not been conjugated to a polypeptide comprising an EGFR targeting moiety.

32-47. (canceled)
